(19) United States
(12) Patent Application Publication
(10) Pub. No.: US 2015/0238215 A1
(43) Pub. Date: Aug. 27, 2015

(54) FLUID EJECTING APPARATUS AND MEDICAL DEVICE USING THE SAME

(71) Applicant: SEIKO EPSON CORPORATION, Tokyo (JP)

(72) Inventors: Hideki KOJIMA, Matsumoto-shi (JP); Hirokazu SEKINO, Matsumoto-shi (JP)

(21) Appl. No.: 14/628,039

(22) Filed: Feb. 20, 2015

(30) Foreign Application Priority Data
Feb. 21, 2014 (JP) 2014-032018

(51) Int. Cl. A61B 17/3203

(52) U.S. Cl. CPC A61B 17/3203; A61B 2017/32032; A61B 2017/00141

(57) ABSTRACT

A fluid ejecting apparatus includes a handpiece, a fluid supply portion, and a bubble generating portion. The handpiece has a fluid chamber filled with a fluid, and a nozzle for ejecting the fluid in the fluid chamber. The fluid supply portion supplies the fluid to the fluid chamber at a predetermined flow rate through a supply tube. The bubble generating portion ejects the fluid from the nozzle by periodically generating a bubble in the fluid chamber. In a case where a frequency when a bubble is generated by the bubble generating portion is f (Hz) and a maximum volume of a bubble when the bubble during one cycle of driving of the bubble generating portion becomes the maximum is V (ml), the fluid supply portion supplies the fluid to the fluid chamber at the predetermined flow rate exceeding V×f (ml/s).

### CROSS-REFERENCE

This application claims the benefit of Japanese Patent Application No. 2014-32018, filed on Feb. 21, 2014. The content of the aforementioned application is incorporated herein by reference in its entirety.

### BACKGROUND

1. Technical Field

The present invention relates to a fluid ejecting apparatus and a medical device using the same.

2. Related Art

In some cases, the fluid ejecting apparatus is used in, for example, medical devices, such as a surgical scalpel or the like, which treat a lesion area by applying a fluid on a biological tissue of the lesion area. JP-A-2008-82202 discloses a fluid ejecting apparatus in which the capacity of a fluid chamber is increased and decreased by driving a piezoelectric device and a pulsating flow (pulse flow) is ejected from an ejection pipe.

The fluid ejecting apparatus is required to have high stability or controllability when ejecting a fluid. Particularly, the fluid ejecting apparatus used in a medical device is required to have an improved comfort of an operator during use in addition to secured stability or controllability when ejecting a fluid at a higher level. Moreover, in the fluid ejecting apparatus, miniaturization, simplification, improvement in operability, low cost, resource saving, easy manufacturing, and the like have been requested.

### SUMMARY

An advantage of some aspects of the invention is to solve at least a part of the problems described above, and the invention can be implemented as the following aspects.

(1) An aspect of the invention provides a fluid ejecting apparatus which ejects a fluid. The fluid ejecting apparatus may include an ejection opening, a fluid chamber, a fluid supply portion, a bubble generating portion, and a control portion. The ejection opening may eject the fluid. The fluid chamber may be communicated with the ejection opening. The fluid supply portion may supply the fluid at a predetermined flow rate to the fluid chamber. The bubble generating portion may eject the fluid within the fluid chamber from the ejection opening by periodically generating a bubble in the fluid chamber. The control portion may control the fluid supply portion and the bubble generating portion. In a case where a frequency when a bubble is generated by the bubble generating portion is f (Hz) and a maximum volume of a bubble when the bubble during one cycle of driving of the bubble generating portion becomes the maximum is V (ml), the control portion may make the fluid supply portion supply the fluid at the predetermined flow rate exceeding V×f (ml/s). According to the aspect of the fluid ejecting apparatus, the fluid is prevented from being insufficient within the fluid chamber and stability in ejecting the fluid is improved.

(2) In the fluid ejecting apparatus according to the aspect described above, the control portion may variably control the frequency f and the maximum volume V, and when the maximum value of the frequency f is fmax (Hz) and the maximum value of the maximum volume V is V1 (ml), the control portion may make the fluid supply portion supply the fluid at the predetermined flow rate exceeding V1×fmax (ml/s). According to this aspect of the fluid ejecting apparatus, the fluid is prevented from being insufficient within the fluid chamber and stability in ejecting the fluid is improved.

(3) In the fluid ejecting apparatus according to the aspect described above, the control portion may make the fluid supply portion supply the fluid at the predetermined flow rate less than V1×2.0×fmax (ml/s). According to this aspect of the fluid ejecting apparatus, the fluid is prevented from being excessively supplied to the fluid chamber and the stability in ejecting the fluid is improved.

(4) In the fluid ejecting apparatus according to the aspect described above, when a fluid with a volume of V1 (ml) is ejected from the ejection opening by one-time driving of the bubble generating portion and when a volume of a fluid which is ejected together with the fluid with the volume of V1 (ml) from the ejection opening by an inertia effect of a fluid is V2 (ml), the control portion may supply the fluid at the predetermined flow rate greater than or equal to (V1+V2)×fmax (ml/s). According to this aspect of the fluid ejecting apparatus, the fluid is prevented from being insufficient in the fluid chamber after the ejection of the fluid.

(5) Another aspect of the invention provides a medical device. The fluid ejecting apparatus described above may be used as the medical device. According to this aspect of the medical device, stability in ejecting the fluid is improved.

The plurality of constituents provided in each aspect of the invention described above are not essential. Moreover, in order to solve a part or all of the problems described above, or to achieve a part or all of the effects described in the present specification, it is possible to appropriately perform modification, deletion, replacement with other new constituents with respect to a part of constituents of the plurality of constituents, or to perform deletion of a part of limited contents. In addition, in order to solve a part or all of the problems described above, or to achieve a part or all of the effects described in the present specification, it is also possible to combine a part or all of the technical features included in an aspect of the invention described above with a part or all of the technical features included in another aspect of the invention described above to make an independent aspect of the invention.

The invention can be implemented in various forms other than the fluid ejecting apparatus. For example, it is possible to implement the invention in forms such as a medical device provided with the fluid ejecting apparatus or a medical system provided with the medical device. In addition, it is possible to implement the invention in forms such as a method of ejecting a fluid, a method of controlling the fluid ejecting apparatus, a computer program for implementing the methods, or a non-temporary recording medium in which the computer program is recorded.

### BRIEF DESCRIPTION OF THE DRAWINGS

The invention will be described with reference to the accompanying drawings, wherein like numbers reference like elements.

FIG. 1 is a schematic view showing a configuration of a fluid ejecting apparatus.

FIG. 2 is a schematic cross-sectional view showing an internal configuration of a handpiece.

FIG. 3 is a schematic view showing an example of a driving signal transmitted to a bubble generating portion from a control portion.

FIGS. 4A to 4E are schematic views showing states within a fluid chamber when one cycle of a driving signal is applied in a time series.

FIG. 5 is an explanatory view for illustrating the volume of a fluid which is pushed out of the fluid chamber due to generation of a bubble.

### DESCRIPTION OF EXEMPLARY EMBODIMENTS

### A. Embodiment

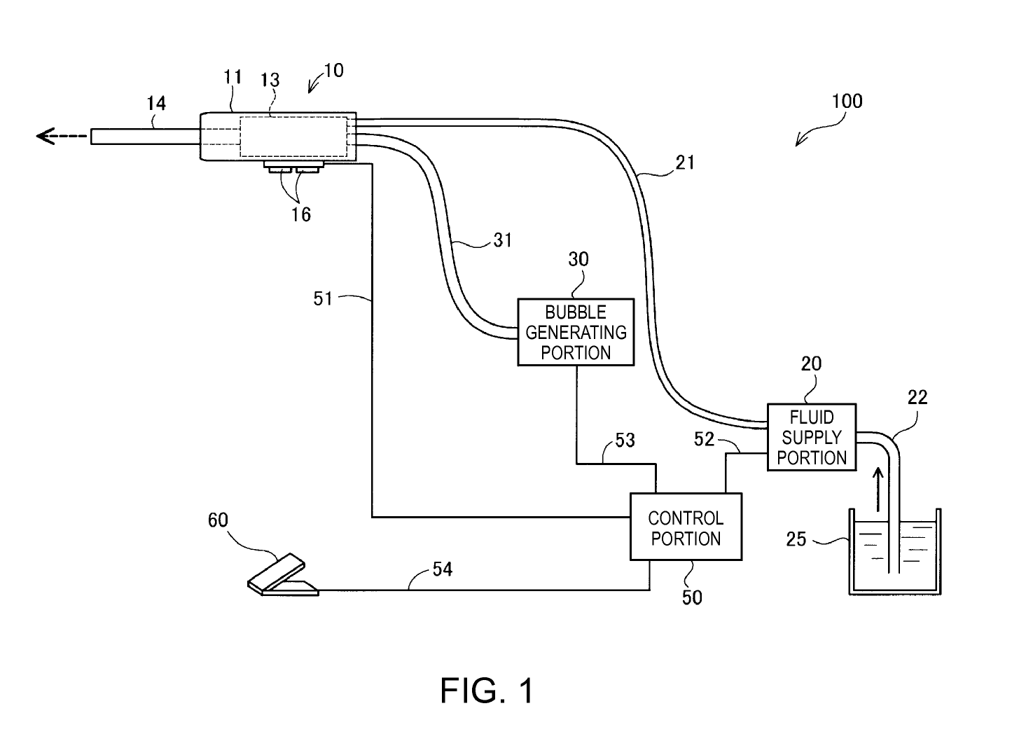

FIG. 1 is a schematic view showing a configuration of a fluid ejecting apparatus 100 according to an embodiment of the invention. The fluid ejecting apparatus 100 is a medical device utilized in medical institutions and has a function of performing incision or excision of a lesion area as a surgical scalpel by ejecting a fluid which is imparted with pulsation to a biological tissue as the lesion area of a patient. In the present specification, the “fluid imparted with pulsation” is called a “pulsating flow” or a “pulse flow”. The fluid ejecting apparatus 100 includes a handpiece 10, a fluid supply portion 20, a fluid container 25, a bubble generating portion 30, and a control portion 50.

The handpiece 10 is a substantially tubular operation tool portion, which is operated by an operator by being held by hand, and ejects a fluid from a tip end thereof in accordance with the operation of the operator as shown by a dashed line arrow in the drawing. The handpiece 10 includes a housing 11, a fluid accommodation portion 13, a nozzle 14, and a condition switching portion 16. The housing 11 is an exterior portion of the handpiece 10 which is configured to be able to be held by an operator, and accommodates the fluid accommodation portion 13 and a portion of the nozzle 14 therein.

The fluid accommodation portion 13 has a fluid chamber (not shown) in which a fluid is accommodated. The fluid accommodation portion 13 is connected to the fluid supply portion 20 through a flexible supply tube 21 made of resin, and is connected to the bubble generating portion 30 through a flexible cable 31, on a rear end side thereof. The nozzle 14 is a tubular member which is communicated with the fluid chamber of the fluid accommodation portion 13 and is connected to the fluid accommodation portion 13 on a tip end side. The nozzle 14 is an ejection pipe which ejects a fluid, and the opening at the tip end thereof functions as an ejection opening. The details of the configuration of the fluid accommodation portion 13 and the mechanism of ejecting the fluid will be described later.

The condition switching portion 16 is provided on a side surface of the housing 11. The condition switching portion 16 is an operation portion with which an operator switches the size of the driving voltage for ejecting a fluid, or the frequency. In the present embodiment, the condition switching portion 16 is configured to have two dial type switches so as to be able to easily select the driving voltage and the frequency through operation with one hand of the operator. The setting values of the driving voltage or the frequency which are set by the operator through the condition switching portion 16 are transmitted to the control portion 50 through a signal line 51.

The fluid supply portion 20 includes a pump (not shown). The fluid supply portion 20 absorbs fluid which is stored in the fluid container 25 through a replenishment tube 22 by driving the pump and supplies the absorbed fluid to the fluid accommodation portion 13 through the above-described supply tube 21. The fluid supply portion 20 is connected to the control portion 50 through a signal line 52. The fluid supply portion 20 starts to supply the fluid to the fluid accommodation portion 13 in accordance with a command from the control portion 50. In the present embodiment, a physiological saline is stored in the fluid container 25. In addition, the replenishment tube 22 is formed of resin. The replenishment tube 22 may also be formed of a material (for example, metal) other than the resin.

The bubble generating portion 30 generates a bubble which becomes a driving force of ejecting a fluid in the fluid in the fluid accommodation portion 13. In the present embodiment, the bubble generating portion 30 generates an electromagnetic wave beam which transmits energy for vaporizing a fluid and generating a bubble. The bubble generating portion 30 includes an oscillator which amplifies light, and generates an optical maser in an infrared region which has a wavelength of 2.06 μm and high directivity, as the electromagnetic wave beam. The electromagnetic wave beam generated by the bubble generating portion 30 is transmitted to the handpiece 10 through the cable 31 constituted by optical fibers. The bubble generating portion 30 periodically generates an electromagnetic wave beam depending on the control signal transmitted from the control portion 50 through a signal line 53.

The control portion 50 is constituted by a microcomputer provided with a main storage device and a central processing unit. The control portion 50 controls the flow rate of supplying a fluid using the fluid supply portion 20 and controls the generation cycle or the intensity of the electromagnetic wave beam using the bubble generating portion 30. The control portion 50 is connected to a foot switch 60, which is a switch operated by an operator using the foot, through a signal line 54. When the operator turns on the foot switch 60 by stepping thereon, the control portion 50 transmits a driving signal to the fluid supply portion 20 and the bubble generating portion 30 to make the fluid supply portion 20 start the supplying of the fluid and to make the bubble generating portion 30 start the generating of a bubble.

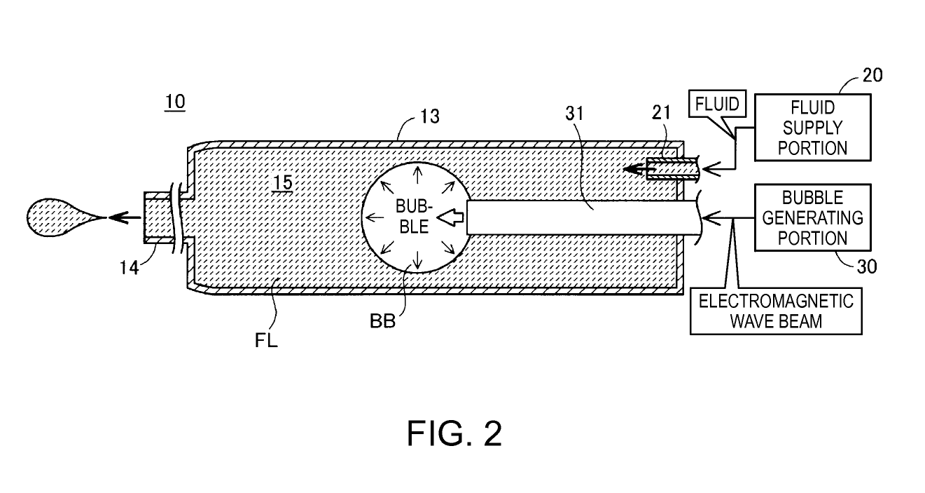

FIG. 2 is a schematic cross-sectional view showing an internal configuration of the handpiece 10. The housing 11, the condition switching portion 16, and a portion of the nozzle 14 are not shown in FIG. 2 for convenience. In addition, the fluid supply portion 20 and the bubble generating portion 30 are shown in FIG. 2 for convenience. The fluid accommodation portion 13 accommodated inside the handpiece 10 has a substantially cylindrical hollow shape, and the fluid chamber 15 filled with a fluid FL is formed therein.

A tip end opening of the supply tube 21 for accepting the supply of the fluid FL from the fluid supply portion 20 is introduced to the fluid chamber 15 on the rear end side (right-hand side on the drawing). In addition, a tip end portion of the cable 31 is introduced to the fluid chamber 15 on the rear end side so as to be able to emit an electromagnetic wave beam which is generated in the bubble generating portion 30 to the fluid FL. The nozzle 14 is connected to the fluid chamber 15 on the tip end side so as to be communicated with the fluid chamber 15. The tip end portion of the cable 31 and the nozzle 14 are disposed such that each of central axes thereof is coincident with a central axis of the fluid chamber 15.

It is desirable that the opening size of the nozzle 14 be greater than that of the supply tube 21 within the fluid chamber 15. Accordingly, the inertance of the nozzle 14 is smaller than that of the supply tube 21, and therefore, when the pressure within the fluid chamber 15 is increased, the fluid FL is easily pushed out of the nozzle 14.

The electromagnetic wave beam (which is shown by a void arrow in the drawing) emitted from the tip end portion of the cable 31 is absorbed by the fluid FL filled in the fluid chamber 15. The fluid FL within the fluid chamber 15 is vaporized by the energy of the absorbed electromagnetic wave beam. Accordingly, a bubble BB is generated within the fluid chamber 15, the pressure within the fluid chamber 15 is instantaneously increased due to rapid growth of the bubble BB, and the fluid FL is ejected from the nozzle 14. When the emission of the electromagnetic wave beam using the bubble generating portion 30 stops, the bubble becomes small and the pressure decreases within the fluid chamber 15. Supplementation of the fluid FL to the fluid chamber 15 through the supply tube 21 is continued at a constant supply flow rate (to be described later), during the series of operations.

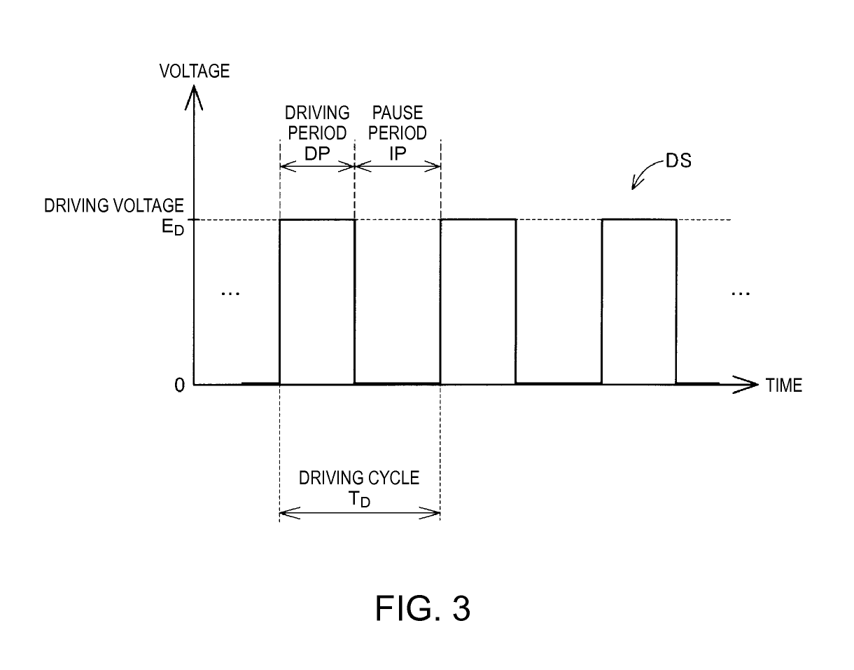

FIG. 3 is a schematic view showing an example of a driving signal DS transmitted to the bubble generating portion 30 from the control portion 50. In FIG. 3, the driving signal DS is represented by a graph of which the longitudinal axis is set to voltage and the horizontal axis is set to time. In the present embodiment, the driving signal DS is constituted by a rectangular wave in which a driving period DP that indicates a maximum voltage E_{D} and a pause period IP that indicates a minimum voltage (0 V) are repeated at a cycle T_{D}. The bubble generating portion 30 continuously repeats the driving and the pause while the driving signal DS is imparted, and periodically generates a bubble within the handpiece 10.

The maximum voltage E_{D} and the cycle T_{D} are respectively values corresponding to the driving voltage and the driving frequency which are set by the condition switching portion 16 (FIG. 1). When the driving voltage is changed by the condition switching portion 16, the maximum voltage E_{D} is changed, and when the driving frequency is changed by the condition switching portion, the cycle T_{D} is changed. Hereinafter, the maximum voltage E_{D} is also called “driving voltage E_{D}^{”} and the cycle T_{D} is also called “driving cycle T_{D}^{”}. In addition, the frequency f_{D} which is obtained from the driving cycle T_{D} (f_{D}=1/T_{D}) is also called “driving frequency f_{D}”. In the present embodiment, the driving voltage E_{D} and the driving frequency f_{D} which can be set by the condition switching portion 16 are as follows.

Driving voltage E_{D}: 0 V to 100 V

Driving frequency f_{D}: 100 Hz to 400 Hz

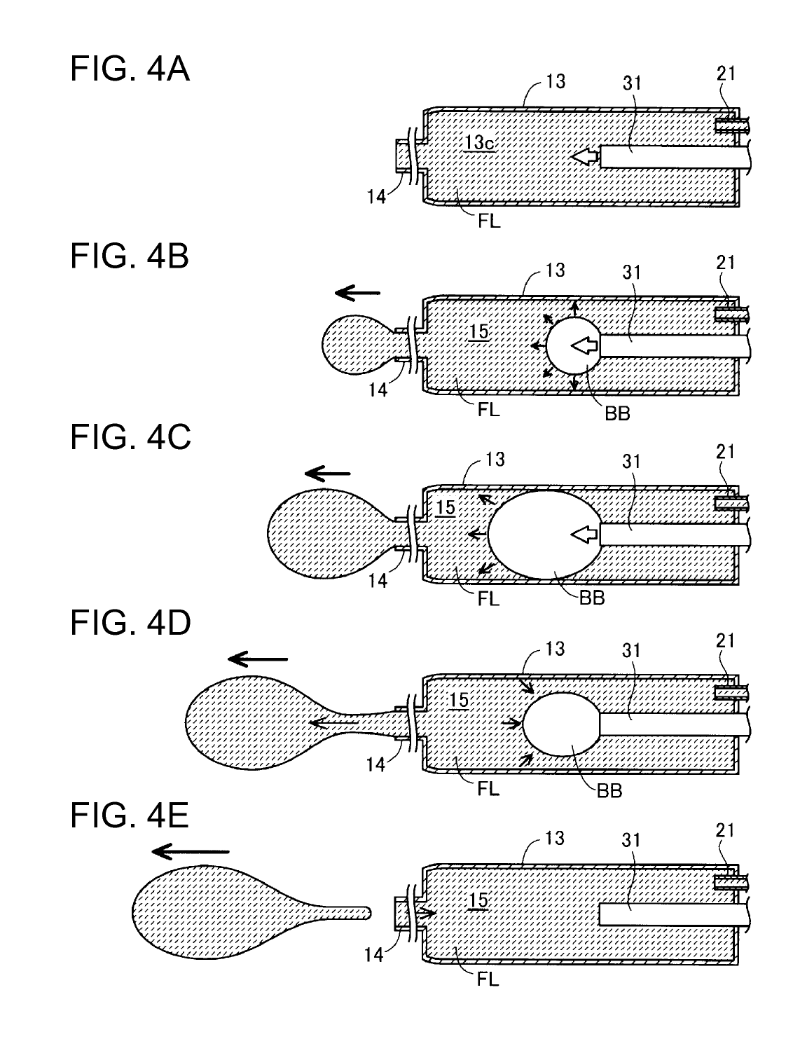

FIGS. 4A to 4E are schematic views showing states within the fluid chamber 15 when one cycle of the driving signal DS is applied. In FIGS. 4A to 4E, the states within the handpiece 10 are shown in each of FIGS. 4A to 4E in a time series. When a voltage as the driving voltage E_{D} is applied to the bubble generating portion 30 in the driving period DP (FIG. 3), the electromagnetic wave beam having energy corresponding to the driving voltage E_{D} is emitted from a tip end portion of the cable 31 (FIG. 4A). Accordingly, a bubble BB is generated in the tip end portion of the cable 31 and the fluid FL starts to be pushed out of the nozzle 14 (FIG. 4B).

The bubble BB grows to a maximum size which is determined in accordance with the driving voltage E_{D} of the fluid supply portion 20 during the driving period DP (FIG. 4C). When the period enters the pause period IP, the emission of the electromagnetic wave beam stops, and therefore, the bubble BB contracts and the pressure within the fluid chamber 15 decreases (FIG. 4D). The mass of the fluid FL pushed out of the nozzle 14 during the driving period DP flies in an opening direction of the nozzle 14 due to the inertia when pushed out of the fluid chamber 15 (FIG. 4E).

When the bubble generating portion 30 is periodically driven by the driving signal DS (FIG. 3), the fluid FL is continuously ejected due to the above-described mechanism, and a pulsating flow of the fluid FL is ejected from the nozzle 14 of the handpiece 10. The greater the driving voltage E_{D} in the driving signal DS is, the greater the pulsating flow ejected from the nozzle 14 is, and therefore, the force of the pulsating flow becomes strong. In addition, the greater the driving frequency f_{D} is, the more the increased pulsation frequency of the pulsating flow is.

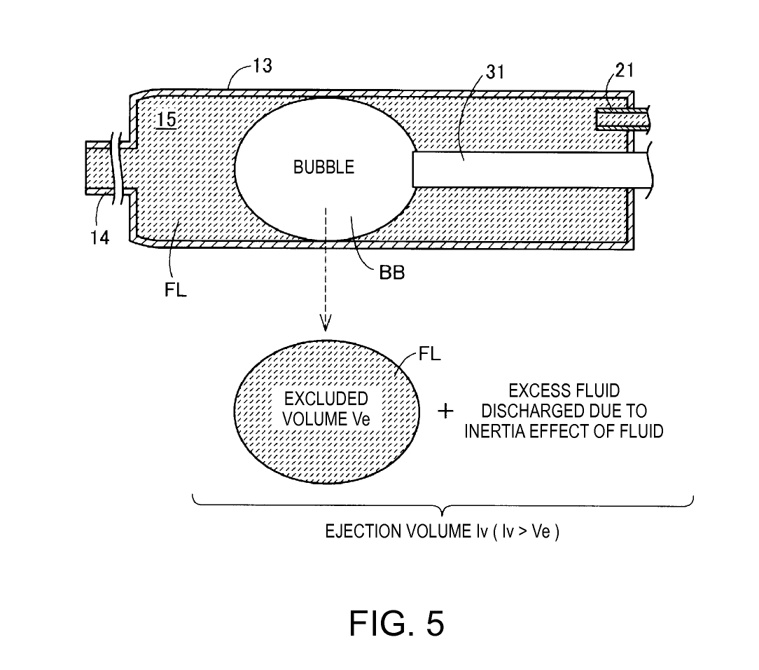

FIG. 5 is an explanatory view for illustrating the volume of the fluid FL which is pushed out of the fluid chamber 15 due to generation of a bubble BB. In FIG. 5, the fluid chamber 15 in a state where the bubble BB has a maximum size in the fluid chamber 15, and the fluid FL having the same volume as that of the bubble BB are comparatively shown. In the present specification, the volume of the fluid FL which is pushed out of the fluid chamber 15 due to the bubble BB during the period of one cycle of the driving signal DS and is excluded from the fluid chamber is also called “excluded volume Ve”. The excluded volume Ve is equivalent to a volume Vb (hereinafter, also called “maximum volume Vb”) of the bubble BB when the size of the bubble BB becomes the maximum in the fluid chamber 15.

The inventor of the invention found that the volume Iv (hereinafter, also called “ejection volume Iv”) of the fluid ejected from the nozzle 14 during the period of one cycle of the driving signal DS is larger than that of the excluded volume Ve (Iv>Ve). As shown in FIG. 4E, when the mass of the fluid FL flies from the nozzle 14, the striated fluid FL which is dragged by the mass of the fluid FL due to the inertia effect of the fluid FL is discharged from the fluid chamber 15. As a result, a fluid FL which is larger than the amount equivalent to the excluded volume Ve is ejected from the nozzle 14 during the period of one cycle of the driving signal DS. Accordingly, the ejection volume Iv is greater than the excluded volume Ve.

Here, as described above, the fluid chamber 15 is supplemented with the fluid FL at a constant supply flow rate by the fluid supply portion 20 while the fluid FL is periodically ejected from the nozzle 14 by the driving signal DS. When the supply flow rate using the fluid supply portion 20 is small, there is a possibility that the fluid FL within the fluid chamber 15 may become insufficient and the ejection of the fluid FL from the nozzle 14 may become unstable.

Therefore, in the fluid ejecting apparatus 100 according to the present embodiment, the control portion 50 makes the fluid supply portion 20 supply the fluid FL to the fluid chamber 15 at a supply flow rate FR to be described below while the pulsating flow is ejected from the nozzle 14.

The excluded volume Ve when the bubble generating portion 30 is driven at a maximum value Vmax of the driving voltage E_{D} and at a maximum value fmax of the driving frequency f_{D} which can be set by the condition switching portion 16 is set to V1 (ml). At this time, the volume of the fluid FL pushed out of the nozzle 14 by the bubble BB from the fluid chamber 15 in one second, that is, the excluded volume Vef (ml/s) per unit time is represented by the following formula (1).

Vef=V1×fmax (1)

In contrast, the ejection volume Iv1 (hereinafter, also called “ejection volume Iv1 per unit time”) of the fluid FL which is ejected from the nozzle 14 in one second becomes greater than the excluded volume Vef per unit time. The control portion 50 makes the fluid supply portion 20 supply the fluid FL to the fluid chamber 15 at a supply flow rate FR exceeding the excluded volume Vef per unit time (FR>Vef).

Accordingly, even when the bubble generating portion 30 is driven at a maximum driving voltage Emax and a maximum driving frequency fmax, the fluid FL within the fluid chamber 15 is prevented from being insufficient. Accordingly, even when the driving voltage E_{D} and the driving frequency f_{D} are set to be smaller than the maximum driving voltage Emax and the maximum frequency fmax by the condition switching portion 16, the fluid FL in the fluid chamber 15 is prevented from being insufficient.

It is desirable that the supply flow rate FR using the fluid supply portion 20 be set to a value greater than or equal to a minimum value FRmin of the flow rate to be described later (FR≧FRmin). The volume of the fluid FL ejected from the nozzle 14 together with the fluid FL of the excluded volume V1 is set to V2 (ml). At this time, the minimum value FRmin of the flow rate is derived from the following formula (2).

FRmin=(V1+V2)×fmax (2)

The total volume V1, V2 is equivalent to the ejection volume Iv (Iv=V1+V2). That is, the amount of an excessively ejected fluid with respect to the excluded volume Ve due to the inertia effect of the fluid FL is reflected in the minimum value FRmin. Accordingly, when the supply flow rate FR is set to be greater than or equal to the minimum value FRmin, the fluid FL is supplied to the fluid chamber 15 at an adequate flow rate in which the amount of the excessively ejected fluid with respect to the excluded volume Ve due to the inertia effect of the fluid is considered. Accordingly, the stability and the controllability of the ejection of the pulsating flow from the nozzle 14 are improved.

In addition, it is desirable that the supply flow rate FR using the fluid supply portion 20 be set to a value less than the maximum value FRmax of the flow rate to be described later (FR<FRmax). It was experimentally confirmed that the volume V2 of the ejected fluid due to the inertia effect of the fluid is smaller than the excluded volume V1. Accordingly, the ejection volume Iv (=V1+V2) which is a volume of the ejection fluid which is ejected from the nozzle 14 when the driving signal DS during one cycle is applied to the bubble generating portion 30 becomes less than V1×2.0 (Iv<V1×2.0). For this reason, it is preferable that the supply flow rate FR of the fluid FL using the fluid supply portion 20 be less than the maximum value FRmax of the supply flow rate which is derived from the following formula (3).

FRmax=V1×2.0×fmax (3)

When the supply flow rate FR is set to a value less than the maximum value FRmax, the fluid FL is prevented from being excessively supplied to the fluid chamber 15, and a continuous flow, which is not accompanied by pulsation caused by the excessive supply of the fluid FL with respect to the fluid chamber 15, is prevented from being ejected from the nozzle 14. The continuous flow of the fluid FL has little contribution to incision, excision, or the like of a lesion area, or becomes a cause of increased fluid remaining in the lesion area, thereby narrowing the surgical field. According to the fluid ejecting apparatus 100 of the present embodiment, it is possible to suppress the generation of the continuous flow, and therefore, an operator can feel a stable feeling of use.

As described above, according to the fluid ejecting apparatus 100 of the present embodiment, when the pulsating flow is ejected from the nozzle 14 of the handpiece 10, the fluid chamber 15 is supplemented with the fluid FL at an adequate supply flow rate FR. Accordingly, the pulsating flow which has a desired strength or pulsation frequency is prevented from not being ejected due to the insufficient fluid FL within the fluid chamber 15. Particularly, when the supply flow rate FR is set to be greater than or equal to the minimum value FRmin, the fluid FL is more reliably prevented from being insufficient within the fluid chamber 15. In addition, when the supply flow rate FR is set to be less than the maximum value FRmax, the fluid FL is prevented from being excessively supplied to the fluid chamber 15 and the continuous flow which is not accompanied by the pulsation is prevented from being ejected from the nozzle 14. Therefore, according to the fluid ejecting apparatus 100 of the present embodiment, the stability and the controllability of the ejection of the fluid FL from the nozzle 14 are improved and an operator can feel a stable feeling of use. However, the constant supply flow rate FR due to the fluid supply portion 20 according to the present embodiment may fluctuate within a range of, for example, ±10% when measured. Even when the pump provided in the fluid supply portion 20 is a roller pump or a plunger pump and when the flow rate is instantaneously fluctuated, this hardly damages the effect of the invention as long as the average flow rate is constant in a macro time cycle.

### B. Modification Examples

### B1. Modification Example 1

In the above-described embodiment, the fluid supply portion 20 supplies a fluid FL to the fluid chamber 15 at a constant supply flow rate FR which is defined based on the maximum values Emax, fmax of the driving voltage E_{D} and the driving frequency f_{D}. In contrast, the supply flow rate FR may not be set based on the maximum values Emax, fmax of the driving voltage E_{D} and the driving frequency f_{D}. The supply flow rate FR may be set to a value exceeding V×f (ml/s) in a case where the frequency at which the bubble generating portion 30 generates a bubble in the fluid chamber 15 is f (Hz) and the maximum volume of a bubble when the bubble becomes the maximum in the fluid chamber 15 during one cycle of driving of the bubble generating portion 30 is V (ml). The maximum volume V is equivalent to the amount of change in the volume of the fluid FL accommodated in the fluid chamber 15 during one cycle of driving of the bubble generating portion 30. In addition, the supply flow rate FR due to the fluid supply portion 20 may be changed depending on the driving voltage E_{D} and the driving frequency f_{D} which are set by the condition switching portion 16. In this case, the control portion 50 may set the supply flow rate FR using a map where a correspondence relation in which the supply flow rate FR is determined with respect to the driving voltage E_{D}, a correspondence relation in which the supply flow rate FR is determined with respect to the driving frequency f_{D}, a correspondence relation in which the supply flow rate FR is determined with respect to a combination of the driving voltage E_{D} and the driving frequency f_{D}, or the like is shown. According to such a configuration, the fluid chamber 15 is adequately supplemented with the fluid FL with an amount in accordance with the amount of the ejected fluid FL. The supply flow rate FR due to the fluid supply portion 20 may be a constant value which is previously set as in the above-described embodiment, or may be a control value which is appropriately changed as in the modification example. The fluid supply portion 20 may supply a fluid FL at a predetermined flow rate including these values.

### B2. Modification Example 2

In the above-described embodiment, the bubble generating portion 30 generates a bubble in the fluid chamber 15 through an optical maser in an infrared region which has a wavelength of 2.06 μm and high directivity. In contrast, the bubble generating portion 30 may generate a bubble in the fluid chamber 15 through optical masers with other wavelengths or electromagnetic wave beams other than the optical maser. Alternatively, the bubble generating portion 30 may generate a bubble in the fluid chamber 15 using units other than those emitting the electromagnetic wave beam. An optical maser in a visible region or an optical maser in an ultraviolet region may be used instead of the optical maser in the infrared region. A coherent microwave may be used as the electromagnetic wave beam other than the optical maser, for example. In this case, a waveguide is employed as the cable 31 instead of the optical fiber. In addition, the bubble generating portion 30 may generate a bubble in the fluid chamber 15 using a microwave or a far-infrared ray which is not coherent. The bubble generating portion 30 may generate a bubble in the fluid chamber 15 through instantaneous heating using an electric heating element such as a resistance heater or a ceramic heater.

### B3. Modification Example 3

In the above-described embodiment, the fluid ejecting apparatus 100 is used as a surgical scalpel. In contrast, the fluid ejecting apparatus 100 may be used as other medical devices. The fluid ejecting apparatus 100 may be used as, for example, a medical device for washing a surgical field. In addition, the fluid ejecting apparatus 100 may be used in devices or apparatuses other than the medical device. The fluid ejecting apparatus 100 may be used as, for example, a cleaning device for removing dirt of an object to which a fluid is to be ejected, a processing device for cutting an object to which a fluid is to be ejected, an image forming apparatus for forming images such as characters or pictures using an ejected fluid, or the like.

### B4. Modification Example 4

In the above-described embodiment, the fluid ejecting apparatus 100 ejects a physiological saline. In contrast, the fluid ejecting apparatus 100 may eject other liquids, for example, pure water or a liquid medicine, which are not harmful for biological tissues instead of the physiological saline. The fluid which the fluid ejecting apparatus 100 ejects may not be a liquid, and for example, a gas or powder may be used. The fluid which the fluid ejecting apparatus 100 ejects may be appropriately selected depending on the usage.

### B5. Modification Example 5

In the above-described embodiment, the range of the driving voltage E_{D} which can be set by the condition switching portion 16 is 0 V to 100 V and the range of the driving frequency f_{D} is 100 Hz to 400 Hz. In contrast, the ranges of the driving voltage E_{D} and the driving frequency f_{D} which can be set by the condition switching portion 16 may be set within ranges other than the above-described ranges. For example, the range of the driving voltage E_{D} may be 10 V to 80 V, and the range of the driving frequency f_{D} may be 80 Hz to 1000 Hz.

### B6. Modification Example 6

In the above-described embodiment, the condition switching portion 16 is provided on the side surface of the handpiece 10. In contrast, the condition switching portion 16 may be provided at positions other than the handpiece 10, for example, on an end surface of the handpiece 10. In addition, the condition switching portion 16 may be provided in sites other than the handpiece 10. For example, the condition switching portion may be provided in the foot switch 60. In this case, the condition switching portion 16 may not be a dial type switch. For example, the condition switching portion may be a slider switch which can be operated by the foot. The condition switching portion 16 may be omitted from the fluid ejecting apparatus 100.

### B7. Modification Example 7

In the present embodiment, the bubble generating portion 30 generates energy for generating a bubble in the outside of the fluid chamber 15 and transmits the energy to the inside of the fluid chamber through the cable 31. In contrast, the bubble generating portion 30 which generates the energy for generating a bubble may be accommodated within the fluid chamber 15.

The invention is not limited to the above-described embodiment, examples, or modification examples and can be implemented in various configurations within the scope not departing from the gist thereof. For example, it is possible to appropriately replace the technical features in the embodiment, the examples, or the modification examples corresponding to the technical features in each of the forms disclosed in the section of Summary with others or to appropriately combine them together in order to solve a part or all of the problems described above, or to achieve a part or all of the effects described above. In addition, it is possible to appropriately delete the technical features which are not described in the present specification as essential features.

What is claimed is:

1. A fluid ejecting apparatus which ejects a fluid, comprising:
an ejection opening which ejects the fluid;
a fluid chamber which is communicated with the ejection opening;
a fluid supply portion which supplies the fluid at a predetermined flow rate to the fluid chamber;
a bubble generating portion which ejects the fluid within the fluid chamber from the ejection opening by periodically generating a bubble in the fluid chamber; and
a control portion which controls the fluid supply portion and the bubble generating portion,
wherein in a case where a frequency when a bubble is generated by the bubble generating portion is f (Hz) and a maximum volume of a bubble when the bubble during one period of driving of the bubble generating portion becomes the maximum is V (ml), the control portion makes the fluid supply portion supply the fluid at the predetermined flow rate exceeding V×f (ml/s).

2. The fluid ejecting apparatus according to claim 1,
wherein the control portion variably controls the frequency f when the bubble is generated in the fluid chamber, and the maximum volume V, and
wherein when the maximum value of the frequency f is fmax (Hz) and the maximum value of the maximum volume V is V1 (ml), the control portion makes the fluid supply portion supply the fluid at the predetermined flow rate exceeding V1×fmax (ml/s).

3. The fluid ejecting apparatus according to claim 2,
wherein the control portion makes the fluid supply portion supply the fluid at the predetermined flow rate less than V1×2.0×fmax (ml/s).

4. The fluid ejecting apparatus according to claim 2,
wherein when a fluid with a volume of V1 (ml) is ejected from the ejection opening by one-time driving of the bubble generating portion and when a volume of a fluid which is ejected together with the fluid with the volume of V1 (ml) from the ejection opening by an inertia effect of a fluid is V2 (ml), the control portion makes the fluid supply portion supply the fluid at the predetermined flow rate greater than or equal to (V1+V2)×fmax (ml/s).

5. A medical device using the fluid ejecting apparatus according to claim 1.

6. A medical device using the fluid ejecting apparatus according to claim 2.

7. A medical device using the fluid ejecting apparatus according to claim 3.

8. A medical device using the fluid ejecting apparatus according to claim 4.
